(19) United States
(12) Patent Application Publication
(10) Pub. No.: US 2021/0179508 A1
(43) Pub. Date: Jun. 17, 2021

(54) DIGESTER COMPRISING A DESULFURIZATION NET COMBINED WITH PENDENT ROPES

(71) Applicant: L'Air Liquide, Société Anonyme pour l'Etude et l'Exploitation des Procédés Georges Claude, Paris (FR)

(72) Inventors: Aude BERTRANDIAS, Paris (FR); David Frimat, Paris (FR); Antonio Trueba, Charenton le Pont (FR); Jacopo Seiwert, Versailles (FR); Jeremy Ollier, Méringnac (FR); Solène Valentin, Voreppe (FR)

(21) Appl. No.: 17/118,268

(22) Filed: Dec. 10, 2020

(30) Foreign Application Priority Data
Dec. 11, 2019 (FR) 1914171

(51) Int. Cl. C05F 17/921; C12M 1/107; C12M 1/00; C12M 1/02

(52) U.S. Cl. CPC C05F 17/921; C12M 41/18; C12M 29/24; C12M 21/04

(57) ABSTRACT

Plant for producing at least partially desulfurized biogas, comprising a biomass digester and/or post-digester, the digester and/or post-digester comprising:
a chamber (1) in which an anaerobic digestion of the biomass takes place, leading to the production of biogas (2) and of digestate (3),
a means for introducing an oxidizing gas,
a desulfurization net (4) placed horizontally and fastened in the upper part of the chamber, and
ropes (5) attached to said desulfurization net and which hang down to the biogas-digestate interface.

### CROSS REFERENCE TO RELATED APPLICATIONS

This application claims the benefit of priority under 35 U.S.C. § 119 (a) and (b) to French Patent Application No. 1914170, filed Dec. 11, 2019, the entire contents of which are incorporated herein by reference.

### TECHNICAL FIELD

The present invention relates to a plant and a process for producing at least partly desulfurized biogas.

### BACKGROUND

Biogas is the gas produced during the decomposition of organic matter in the absence of oxygen (anaerobic fermentation), also known as methanisation. The decomposition may be natural, as observed in swamps or in household rubbish dumps, though the production of biogas may also result from the methanisation of wastes in a dedicated reactor, under controlled conditions, known as a methaniser or digester, and then in a post-digester, which is similar to the digester and allows the methanisation reaction to be extended.

Biomass refers to any group of organic matters that can be converted into energy through this methanisation process: for example, treatment plant sludges, manures/slurries, agricultural residues, food wastes, etc.

The digester, namely the reactor dedicated to the methanisation of the biomass, is a closed vessel, heated or not (operated at a set temperature, between the ambient temperature and 55° C.), the contents of said vessel, composed of the biomass, being mixed, continuously or sequentially. The conditions in the digester are anaerobic, and the biogas generated is found in the headspace of the digester (gas space), from where it is withdrawn. Post-digesters are similar to digesters.

Owing to its main constituents—methane and carbon dioxide—biogas is a powerful greenhouse gas; at the same time, it also constitutes a source of renewable energy, which is appreciable in the context of the increasing scarcity of fossil fuels.

Biogas contains predominantly methane (CH₄) and carbon dioxide (CO₂), in proportions which can vary according to the substrate and to the way in which the biogas is obtained; however it may also contain, in smaller proportions, water, nitrogen, hydrogen sulfide (H₂S), oxygen, and also other organic compounds, in the form of traces, including H2S, between 10 and 50 000 ppmv.

Depending on the organic matter which has undergone decomposition, and on the techniques used, the proportions of the components differ; on average, however, biogas comprises, on a dry gas basis, from 30% to 75% of methane, from 15% to 60% of CO₂, from 0% to 15% of nitrogen, and from 0% to 5% of oxygen and trace compounds.

Biogas is made use of economically in various ways. It can, after a gentle treatment, be exploited close to the production site in order to supply heat, electricity or a mixture of both (cogeneration); the high carbon dioxide content reduces its calorific value, increases the costs of compression and of transportation and limits the economic advantage of making use of it economically to this use nearby.

More intensive purification of biogas allows it to be more widely used; in particular, intensive purification of biogas makes it possible to obtain a biogas which has been purified to the specifications of natural gas and which can be substituted for the latter; biogas thus purified is known as “biomethane”. Biomethane thus supplements natural gas resources with a renewable part produced within territories; it can be used for exactly the same uses as natural gas of fossil origin. It may supply a natural gas network or a vehicle filling station; it may also be liquefied for storage in the form of liquefied natural gas (bioLNG), etc.

Depending on the composition of the biomass, the biogas produced in the digestion contains hydrogen sulfide (H₂S) in amounts of between 50 and 50 000 ppm.

Irrespective of the final commercial destination of the biogas, it proves to be vital to remove hydrogen sulfide, which is a toxic and corrosive impurity, Moreover, if the use of the biogas involves purifying it for injection of biomethane into the natural gas network, there are strict specifications limiting the permitted quantity of H₂S.

A number of methods exist for removing H₂S and are more or less widespread (beds of activated carbon, addition of iron compounds, physical or chemical absorption, water washing, biofilters, etc.). Removal is accomplished primarily by adsorption on a bed of activated carbon, outside the digester. In an increasing number of digesters, H2S abatement is also accomplished in part by injecting air/enriched air/O₂ into the gas space of the digester, so constituting an in situ solution. With injection into the gas space at a low rate, solid sulfur is formed from the H₂S and O₂ (eq. (1)), as performed by sulfur-oxidizing bacteria, e.g. Thiobacillus. With a high rate of O₂ injected, the mixture is acidified (eq. (2)). The target reaction is therefore reaction (1).

H₂S+0.5O₂→S+H₂O (1)

H₂S+2O₂→SO₄²⁻+2H+ (26)

The amounts of O2 which need to be injected in practice are different from those expected from the stoichiometry of eq. (1): doses of 0.3%-3% O₂ relative to the biogas generated are most usually recommended, with doses of up to 12% being sometimes stated.

Presently, the in situ injection of air/enriched air/O₂ is not optimized, and the beds of activated carbon must therefore be maintained in order to remove all of the H₂S.

From this basis, one problem which arises is that of providing an improved plant promoting greater removal of H₂S.

### SUMMARY

A solution of the present invention is a plant for producing at least partially desulfurized biogas, comprising a biomass digester and/or post-digester, the digester and/or post-digester comprising:
a chamber 1 in which an anaerobic digestion of the biomass takes place, leading to the production of biogas 2 and of digestate 3,
a means for introducing an oxidizing gas,
a desulfurization net 4 placed horizontally and fastened in the upper part of the chamber, and
ropes 5 attached to said desulfurization net and which hang down to the biogas-digestate interface.

It is noted that the biogas-digestate interface corresponds to the gas-liquid interface.

### BRIEF DESCRIPTION OF THE DRAWINGS

For a further understanding of the nature and objects for the present invention, reference should be made to the following detailed description, taken in conjunction with the accompanying drawings, in which like elements are given the same or analogous reference numbers and wherein:

FIG. 1 is a diagram of a cross-sectional view of the inside of the chamber of the digester.

### DETAILED DESCRIPTION OF PREFERRED EMBODIMENTS

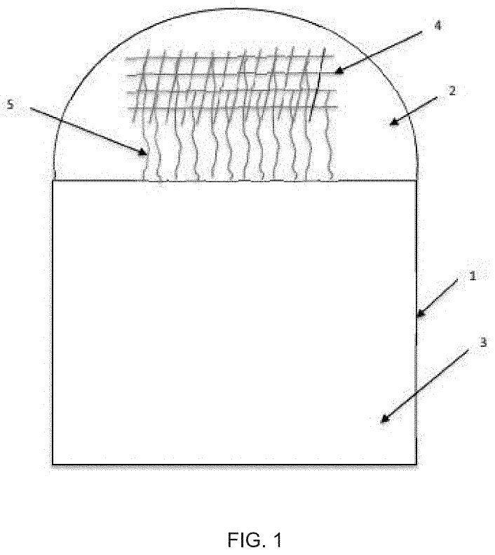

FIG. 1 is a diagram of a cross-sectional view of the inside of the chamber of the digester.

On the inside of the chamber of the digester, when the hydrogen sulfide reacts with oxygen, sulfur attaches itself in particular to the desulfurization net and to the ropes at the gas-liquid interface.

The ropes make it possible to offer an additional surface to the sulfur-oxidizing bacteria, e.g. Thiobacillus, in order to remove sulfur at the location where the hydrogen sulfide is most concentrated, i.e. close to the gas-liquid interface. A better sulfur removal is thus obtained.

Depending on the case, the plant according to the invention may have one or more of the features below:
the ropes are made of polyethylene.
the ropes have a diameter of between 1 and 50 mm, preferably between 2 and 10 mm.
the ropes have a tensile strength of between 50 and 50 000 kg, preferably between 100 and 500 kg.
the ropes withstand temperatures of between −30° C. and 70° C., preferably between 10° C. and 60° C.
the net-rope assembly occupies between 0.1% and 3% of the gas space located between the digestate and the top end of the chamber.
porous blocks are attached to the net and/or the ropes.
the porous blocks comprise iron oxide and/or biochar and/or an activated carbon. Generally, the porous blocks may comprise any known hydrogen sulfide adsorbent.
the ropes are made of polyethylene.

The net could be of octagonal shape. It could also be fastened in the chamber by attaching the net to hooks fastened to the internal wall of the chamber.

A further subject of the present invention is a process for producing at least partially desulfurized biogas, using a plant according to the invention, comprising::
injecting biomass into the digester;
injecting an oxidizing gas at the top of the digester; and
mixing the biomass.

Note that the oxidizing gas might be oxygen or air or enriched air. Enriched air refers to air having a higher oxygen content than the oxygen content normally present in air.

As explained above, the sulfur attaches itself to the net and the ropes. After a certain period of time, the solid sulfur generated falls into the digestate and is discharged therewith.

The solution according to the invention makes it possible to obtain a biogas stream comprising less than 200 ppm of hydrogen sulfide.

The invention makes it possible to reduce the costs of purifying biogas by removal of hydrogen sulfide effectively, by increasing the reactivity of the oxygen already injected with the sulfur-containing products, by creating an additional reaction surface within the gas space of the digester, with no need for complex engineering.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

While embodiments of this invention have been shown and described, modifications thereof may be made by one skilled in the art without departing from the spirit or teaching of this invention. The embodiments described herein are exemplary only and not limiting. Many variations and modifications of the composition and method are possible and within the scope of the invention. Accordingly the scope of protection is not limited to the embodiments described herein, but is only limited by the claims which follow, the scope of which shall include all equivalents of the subject matter of the claims.

What is claimed is:

1. A plant for producing at least partially desulfurized biogas, comprising a biomass digester and/or post-digester, the digester and/or post-digester comprising:
a chamber (1) in which an anaerobic digestion of the biomass takes place, leading to the production of biogas (2) and of digestate (3),
a means for introducing an oxidizing gas,
a desulfurization net (4) placed horizontally and fastened in the upper part of the chamber, and
ropes (5) attached to said desulfurization net and which hang down to the biogas-digestate interface.

2. The plant according to claim 1, wherein the ropes are made of polyethylene.

3. The plant according to claim 1, wherein the ropes have a diameter of between 1 and 50 mm, preferably between 2 and 10 mm.

4. The plant according to claim 1, wherein the ropes have a tensile strength of between 50 and 50 000 kg, preferably between 100 and 500 kg.

5. The plant according to claim 1, wherein the ropes withstand temperatures of between −30° C. and 70° C., preferably between 10° C. and 60° C.

6. The plant according to claim 1, wherein the net-rope assembly occupies between 0.1% and 3% of the gas space located between the digestate and the top end of the chamber.

7. The plant according to claim 1, wherein porous blocks are attached to the net and/or the ropes.

8. The plant according to claim 7, wherein the porous blocks comprise iron oxide and/or biochar and/or an activated carbon.

9. A process for producing at least partly desulfurized biogas, using a plant according to claim 1, which comprising:
injecting biomass into the digester;
injecting an oxidizing gas at the top of the digester; and
mixing the biomass.

10. The process according to claim 9, wherein the oxidizing gas is oxygen or air or enriched air.
